SEQUENCE LISTING (6 sequences)

SEQ ID NO: 1 - DNA, 32 bp, Artificial Sequence
  Synthetic
gtctctggatccatcgacatgctgggaatagg

SEQ ID NO: 2 - DNA, 30 bp, Artificial Sequence
  Synthetic
ggttgtggatccaggatggaaatccgcagg

SEQ ID NO: 3 - DNA, 1614 bp, Equus caballus
ttgtggccacaggatggaaatccgcaggcgttctgtcagacacctaatctctctcctccttttcttgctctactcagagacagcctgccaccctttggggaagagaccctgcaagatgcaagccttcagaatctgggatgttaaccagaagaccttctacatgaggaataaccaactagttgctggatacttgcaagaatcaaatactaaattacaagagaagatagatgtggtgcccattgagcctgatgctctattcctgggactccatgggaggaagctgtgcctggcctgtgtcaagtctggtgatgagattaggttccaattggaggcagttaacatcactgacctgagcaagaacaaggaggagaacaagcgcttcaccttcatccgctcaaacagtggccccaccaccagcttcgagtctgccgcctgccctggctggttcctctgcacggcgcaggaggcagaccggcccgtcagcctcaccaacaagcccaaagagtccttcatggtcaccaagttctacctccaggaggaccagtagaactgcccgtagctccgcttcccctattcccagcatgtcgatgcatccagagactgcctctccaccccagggtctcctggggctctgaggagcagccttggcagggtgggccctcagaaggaggtacacgagccctcgtaacaggactctgtctccagcctcctcagctatccaccctccatgctgcttccaccatggtcctttctaaagtgcagctcaaaccacggccctacttaaagcccttcagtgtcgtctgtaccttcaggataaaatccgggccacctggccagcctggatgccttctgctctcgtctgtcaagtgttcgttgtccctcaccccactgtccctgcccgagatccctcaggccactcactggccccccaccaatggctctcataccttgttttggaatctgatgctgttctgtagggaagacttttagagtctgtggcaaaatggaaaataagaatttcatgaaactttctaaagccagctttatccaatttgaaggagagtcctttatttggagattatttcctttttgcaaaggggtggggatcaaaatattcctgtgtttgtgaagtgatagtgaagggaggttcccttgttagtgtccattcttgtttttgtaataccctaaccggtaaaaatgaacagttagtgtactatgtttgtcccttattcttttctttgtgaaacgtttcctgtaagtctggacccactgcccgggcctgagcaccacctccattgcagacctttcacagctgcccacagtgctttcccctcccatcaaaggttcccctgctcccacggcacagaacaaatgtggctcctccaaggcctttcctgatctcccgggaggaatgaatcgctccttgaccattttagcacttctgacgctctgaaacttgtttgaaaggtggttatgcctctgtctgtctcctgccccaaactgtgagctcctggaagcagggacatgactggcatgtgtctcagcttcccccagggccaagcacatggcctgttttacaataaaaccttgaaattcaaaaaaaaaaaaaaaaaa

SEQ ID NO: 4 - DNA, 531 bp, Equus caballus
atggaaatccgcaggcgttctgtcagacacctaatctctctcctccttttcttgctctactcagagacagcctgccaccctttggggaagagaccctgcaagatgcaagccttcagaatctgggatgttaaccagaagaccttctacatgaggaataaccaactagttgctggatacttgcaagaatcaaatactaaattacaagagaagatagatgtggtgcccattgagcctgatgctctattcctgggactccatgggaggaagctgtgcctggcctgtgtcaagtctggtgatgagattaggttccaattggaggcagttaacatcactgacctgagcaagaacaaggaggagaacaagcgcttcaccttcatccgctcaaacagtggccccaccaccagcttcgagtctgccgcctgccctggctggttcctctgcacggcgcaggaggcagaccggcccgtcagcctcaccaacaagcccaaagagtccttcatggtcaccaagttctacctccaggaggaccag

SEQ ID NO: 5 - protein, 177 aa, Equus caballus
MEIRRRSVRHLISLLLFLLYSETACHPLGKRPCKMQAFRIWDVNQKTFYMRNNQLVAGYLQESNTKLQEKIDVVPIEPDALFLGLHGRKLCLACVKSGDEIRFQLEAVNITDLSKNKEENKRFTFIRSNSGPTTSFESAACPGWFLCTAQEADRPVSLTNKPKESFMVTKFYLQEDQ

SEQ ID NO: 6 - DNA, 603 bp, Artificial Sequence
  PCR amplified
ggttgtggatccaggatggaaatccgcaggcgttctgtcagacacctaatctctctcctccttttcttgctctactcagagacagcctgccaccctttggggaagagaccctgcaagatgcaagccttcagaatctgggatgttaaccagaagaccttctacatgaggaataaccaactagttgctggatacttgcaagaatcaaatactaaattacaagagaagatagatgtggtgcccattgagcctgatgctctattcctgggactccatgggaggaagctgtgcctggcctgtgtcaagtctggtgatgagattaggttccaattggaggcagttaacatcactgacctgagcaagaacaaggaggagaacaagcgcttcaccttcatccgctcaaacagtggccccaccaccagcttcgagtctgccgcctgccctggctggttcctctgcacggcgcaggaggcagaccggcccgtcagcctcaccaacaagcccaaagagtccttcatggtcaccaagttctacctccaggaggaccagtagaactgcccgtagctccgcttcccctattcccagcatgtcgatggatccagagac

(19) United States
(12) Patent Application Publication
(10) Pub. No.: US 2003/0091536 A1
(43) Pub. Date: May 15, 2003

(54) In vivo treatment of joint disease using interleukin-1

(75) Inventors: David D. Frisbie, Fort Collins, CO (US); C. Wayne McIlwraith, Fort Collins, CO (US)

Correspondence Address: Janelle D. Waack, HOWREY SIMON ARNOLD & WHITE LLP, 750 Bering Drive, Houston, TX, 77057-2198, US

(73) Assignee: Colorado State University Research Foundation

(21) Appl. No.: 10/092,849

(22) Filed: Mar. 7, 2002

Related U.S. Application Data
(60) Provisional application No. 60/152,707, filed on Sep. 7, 1999.

(30) Foreign Application Priority Data
Sep. 6, 2000 (US) PCT/US00/40823

(51) Int. Cl. A61K 48/00; C12N 15/86; A61K 38/20

(52) U.S. Cl. 424/93.2; 424/85.2; 514/44; 435/456

(57) ABSTRACT

Adenovirus particles encoding an interleukin-1 receptor antagonist were found to be effective in treating joint disease over an extended period of time when administered directly via arthrocentesis.

Administration of the adenovirus particles resulted in an increase in interleukin-1 receptor antagonist protein concentration, increased influx of white blood cells, increased percentage of neutrophils, and a lower percentage of mononuclear cells when compared to control experiments.

### FIELD OF THE INVENTION

The invention relates to the in vivo treatment of joint disease using gene therapy. In particular, in vivo administration of vectors encoding interleukin-1 receptor antagonists is disclosed.

### BACKGROUND OF THE INVENTION

Joint disease is a severe mobility and career modifying disease in man and horses (Robert Davis, Horses give a leg up to human knee research, U.S.A. Today, Apr. 6, 1999, at 10D). The etiopathogenesis of joint disease is not clearly understood, however, may mediators that lead to degeneration within the joint environment have been identified. Cytokines, especially interleukin-1 and tumor necrosis factor, have been defined as initiating mediators of a degenerative cascade that propagates joint disease. Through the use of anti-cytokine proteins it has been demonstrated that blocking these molecules can significantly alter the course of joint disease. These studies have mainly been performed on experimentally created joint disease. Furthermore, both the effects on naturally occurring disease and long-term outcomes of joint disease have yet to be adequately studied using anti-cytokine therapies.

Over the last decade, gene transfer has become an accepted and even preferred method of continuous protein expression targeting certain disease conditions although the optimal vector for use in gene transfer is still undetermined. Early in the genesis of gene transfer protocols the use of retroviral vectors predominated for various practical and safety reasons. Some of these reasons include the ability to harvest cells, transduce them in vitro, and after safety testing, introduce the altered cells into the recipient.

Two basic methodologies are utilized to transfer vectors into target tissues. In ex vivo gene transfer, cells are collected from the patient or host and grown in the laboratory. During a finite culture period the therapeutic genes are transferred into the cells via a vector. Once tested for the correct behavior or in many cases protein production, the “transduced” cells are re-implanted in the patient. This methodology is currently quite prevalent in human gene therapy trials, (Martin, P. A. and Thomas, S. M., Hum. Gene Ther., 9: 87-114, 1998) partially because the administrator has the ability to control and test the cells prior to re-introduction into the patient, thus giving the utmost consideration to safety issues.

In vivo methodologies refer to the direct transfer of the vector to the target tissues in situ. Although this method does not allow extensive safety testing, its utility and ease of application are very attractive, and for these reasons many planned gene therapy protocols are utilizing this methodology. (Martin, P. A. and Thomas, S. M., Hum. Gene Ther., 9: 87-114, 1998)

Vectors are classified generally as either non-viral (synthetic) or viral. Non-viral vectors typically refer to synthetic molecules that facilitate the uptake of DNA into cells by condensing the DNA with lipids, peptides, proteins, inactivated virus particles, crystals of calcium phosphate, or coated microprojectiles. Viral vectors are viruses from which the viral genes have been removed to allow insertion of the therapeutic gene(s), and the viral/vector has usually been rendered incapable of replicative spread. As a general rule viral vectors give much higher efficiencies of gene delivery than non-viral vectors. Many well characterized viruses have been explored for use as vectors, but to date retroviral and adenoviral vectors have proven the most useful. (Verma, J. M. and Somia, N., Nature 389: 239-242, 1997; Martin, P. A. and Thomas, S. M., Hum. Gene Ther., 9: 87-114, 1998; Robbins, P. D., et al., Trends Biotech., 16: 35-40, 1998).

Retroviral vectors integrate their DNA (including a gene sequence of interest) into the chromosomal DNA of the target cells, ensuring gene transfer to the target cell progeny. However, retroviral vectors only transduce (infect) dividing cells, making them useful in certain situations but less useful in others. If for example the target population of cells were gastrointestinal epithelium which divide very rapidly, a retroviral vector could be quite useful in a gene transfer protocol. Adenoviral vectors can transduce non-dividing cells allowing them to be used on a larger range of cell types. If the goal of a protocol was to transduce slow or non-dividing cells, such as neurons, an adenoviral vector would be a superior choice. (Robbins, P. D., et al., Trends Biotech., 16: 35-40, 1998)

In addition to work based on the engineering of equine gene vaccines, (Guo, P. X., et al., J. Virol., 64: 2399-2406, 1990) another active avenue of equine gene therapy is targeting treatments for joint disease. Based on a collaboration between the University of Pittsburgh School of Medicine (Departments of Orthopaedic Surgery and Molecular Genetics) and Colorado State University (Equine Orthopaedic Research Laboratory), notable recent advances have been made. The sequence coding for the equine (Equus caballus) interleukin-1 receptor antagonist protein (IRAP), an anti-arthritic protein, has been identified and cloned, (Howard, R. D., et al., Am. J. Vet. Res., 59: 712-716,1998; GenBank Accession No. U92482, SEQ ID NO: 3).

Traditional joint disease therapy has been partially hindered by the relative inability to target therapeutic agents directly to the joint when they are not administered intraarticularly (enteral, parenteral, and intramuscular routes of administration; Levick, J. R., American Physiology Society, 4: 917-947, 1994). Although intraarticular (IA) administration avoids these limitations, the half-life of most commonly used agents administered directly into the joint space is short, and frequent IA injections are needed to sustain biologic activity for prolonged treatment periods (Mankin, H. J. and Radin, E. L., Structure and function of joints. Arthritis and allied conditions: A textbook of rheumatology. D. J. McCarty. Philadelphia, Pa., Lea & Febiger: 181-210, 1993). In addition, many drugs cannot be given by direct IA injection.

In an attempt to circumvent some of these limitations, gene transfer is being investigated in numerous species, not to deliver the protein but rather the gene to the joint space. Gene transfer provides an excellent alternative to conventional therapy whereby a single IA injection can result in local production of a specific therapeutic protein within diseased joint(s) for a prolonged period of time (Otani, K., et al., J. Immunol., 156: 3558-3562, 1996, demonstrating suppression of arthritis in rabbits via ex vivo gene therapy). Once an ideal vector has been chosen, the next step is selecting the appropriate gene sequence to express. This decision is often based on the current knowledge of disease pathogenesis and availability of gene sequences.

Results from joint disease research suggest that the cytokines IL-1 and TNF modulate the synthesis of metalloproteinases by both chondrocytes and synovial cells, and are largely responsible for the mediation of joint disease (Arend, W. P. and Dayer, J. M., Arthritis and Rheumatism, 33: 305-315, 1990; Arend, W. P. and Dayer, J. M., Arthritis and Rheumatism, 38: 151-160, 1995, Wood, D. D., et al., Arthritis and Rheumatism, 28: 853-862, 1996). Identification of various forms of cytokine inhibitors raised the possibility for their therapeutic use. Clinical trials in using the protein forms of cytokine modulating agents, (receptor antagonists, soluble forms of receptors, and monoclonal antibodies against cytokines) have shown significant therapeutic benefits (Wood, D. D., et al., Arthritis and Rheumatism, 28: 853-862, 1996; Goodwin, R. G., et al., Mol. Cell Biol., 11: 3020-3026, 1991; Loetscher, H., et al., Cell, 61: 351-359, 1990; Schall, T. J., et al., Cell, 61: 361-370, 1990; Lewthwaite, J., et al., J. Rheumatol., 21: 467-472, 1994; Campion, G. V., Ann. Rheum. Dis., 53: 485-487, 1994). Furthermore, using both ex vivo and in vivo gene transfer, the human IRAP gene sequence has shown beneficial effects in joint disease models using both dogs and laboratory animals. (Roessler, B. J., et al., Hum. Gene Ther., 6: 307-316, 1995; Hung, G., et al., Gene Therapy, 1: 64-69, 1994; Otani, K., et al., J. Immunol., 156: 3558-3562, 1996; Pelletier, J. P., et al., Arthritis and Rheumatism 40: 1012-1019, 1997). Human IRAP has additionally been shown to suppress experimental arthritis in rats via ex vivo gene transfer (Makarov, S. S. et al., Proc. Natl. Acad. Sci. U.S.A. 93: 402-406, 1996).

There exists a need for novel and improved methods for the treatment of joint disease in mammals. Especially beneficial would be the development of a clinically relevant treatment for joint disease.

### SUMMARY OF THE INVENTION

Direct administration of vectors encoding the interleukin-1 receptor antagonist protein (IRAP) to joint tissue is disclosed as a favorable method for treating joint disease. A preferred embodiment involves the intraarticular injection of adenovirus vectors into the joint space of an arthritic horse leading to long term production of the interleukin-1 receptor antagonist protein, and an accompanying improvement in the condition of the joint.

Direct administration of vectors encoding the interleukin-1 receptor antagonist protein (IRAP) to joint tissue may also be performed as a preventitive or prophylactic measure.

### DESCRIPTION OF THE FIGURES

The following figures form part of the present specification and are included to further demonstrate certain aspects of the present invention. The invention may be better understood by reference to one or more of these drawings in combination with the detailed description of specific embodiments presented herein.

| Figure | Description |
|---|---|
| 1 | The concentration of IRAP measured in the media 48 hours post transduction of |
| | equine synoviocytes with various MOI concentration of eq-adIRAP vector. The |
| | IRAP concentrations are normalized to 1 × 10⁶ cells, and different letters |
| | represent a statistical difference between groups using a P-value < 0.05. |
| 2 | The concentration of PGE₂ measured in the media by treatment group. The No |
| | IL-1 + No IRAP treatment group were cells not exposed to IL-1α and non- |
| | transduced incubated for 48 hours. The No IL-1 + IRAP treatment group were |
| | cells not exposed to IL-1α but transduced with eq-adIRAP. The IL-1 + No |
| | IRAP treatment group were exposed to IL-1α for 48 hours but non-transduced. |
| | The IL-1 + IRAP treatment group were cells exposed to IL-1α for 48 hours and |
| | transduced with eq-adIRAP (10 MOI). Different letters indicate a statistical |
| | difference between groups (P-value < 0.05). |
| 3 | The concentration of IRAP measured in synovial fluid post transduction with |
| | various doses of eq-adIRAP vector or placebo treatment (0 × 10¹⁰ particles). |
| | Asterisks (*) denotes a statistical difference (P-value < 0.05) in the data point |
| | compared to placebo treatment at that time period. |
| 4 | The concentration of IRAP measured in synovial fluid post transduction with |
| | either 0 or 50 × 10¹⁰ particles/joint of the eq-adIRAP vector. Asterisks (*) |
| | denotes a statistical difference (P-value < 0.05) in the data point compared to |
| | placebo treatment at days 0-14. |
| 5 | The WBC counts measured in synovial fluid post transduction with various |
| | doses of eq-adIRAP vector or placebo treatment (0 × 10¹⁰ particles). Asterisks |
| | (*) denotes a statistical difference (P-value < 0.05) in the data point compared |
| | to placebo treatment at that time period. |
| 6 | The percent neutrophils comprising the WBC differential count from synovial |
| | fluid plotted by various doses of eq-adIRAP vector or placebo treatment (0 × |
| | 10¹⁰ particles). Asterisks (*) denotes a statistical difference (P-value < 0.05) |
| | in the data point compared to placebo treatment at that time period. |
| 7 | The percent lymphocytes comprising the WBC differential count from synovial |
| | fluid plotted by days post transduction (averaged over all concentrations). |
| | Asterisks (*) denotes a statistical difference (P-value < 0.05) in the data point |
| | compared to placebo treatment at that time period. |

### DESCRIPTION OF THE SEQUENCE LISTINGS

The following sequence listings form part of the present specification and are included to further demonstrate certain aspects of the present invention. The invention may be better understood by reference to one or more of these sequences in combination with the detailed description of specific embodiments presented herein.

| SEQ ID NO: | Description |
|---|---|
| 1 | PCR primer Horse 3 |
| 2 | PCR primer Horse 5 |
| 3 | Equine IRAP nucleotide mRNA sequence |
| 4 | Equine IRAP nucleotide coding sequence, nucleotides |
| | 14-544 of SEQ ID NO:3 |
| 5 | Equine IRAP amino acid sequence, encoded by |
| | SEQ ID NO:4 |
| 6 | PCR amplified Equine IRAP nucleotide coding sequence |

### DEFINITIONS

The following definitions are provided in order to aid those skilled in the art in understanding the detailed description of the present invention.

“Amplification” refers to increasing the number of copies of a desired nucleic acid molecule. Amplification routinely refers to the polymerase chain reaction (PCR).

“Arthrocentesis” refers to the addition to or replacement of fluid in a mammalian joint through a needle inserted into the synovial capsule. Arthrocentesis may be formed to remove fluid for therapeutic or analytical purposes, or to add a fluid to the joint.

The phrases “coding sequence”, “open reading frame”, and “structural sequence” refer to the region of continuous sequential nucleic acid triplets encoding a protein, polypeptide, or peptide sequence.

“Codon” refers to a sequence of three nucleotides that specify a particular amino acid.

The term “expression” refers to the transcription of a gene to produce the corresponding mRNA and translation of this mRNA to produce the corresponding gene product, i.e., a peptide, polypeptide, or protein.

“Ex vivo” refers to preparation of one or more transgenic cells in vitro, followed by administration of the cells in vivo to an animal.

The term “gene” refers to chromosomal DNA, plasmid DNA, cDNA, synthetic DNA, or other DNA that encodes a peptide, polypeptide, protein, or RNA molecule, and regions flanking the coding sequence involved in the regulation of expression.

“Identity” refers to the degree of similarity between two nucleic acid or protein sequences. An alignment of the two sequences is performed by a suitable computer program. A widely used and accepted computer program for performing sequence alignments is CLUSTALW vl.6 (Thompson, et al. Nucl. Acids Res., 22: 4673-4680, 1994).

“In vitro” refers to in the laboratory.

“In vivo” refers to in a living organism.

“IRAP” refers to interleukin-1 receptor antagonist protein. “h-RAP” refers to human IRAP, “eq-IRAP” refers to equine IRAP, and “adIRAP” refers to an adenovirus vector encoding IRAP.

“Joint disease” refers to diseases which restrict the mobility of joints in mammals. Joint disease includes osteoarthritis and rheumatoid arthritis.

“Mutation” refers to any change or alteration in the sequence of a gene. Several types exist, including point, frame shift, and splicing.

“Nucleic acid” refers to deoxyribonucleic acid (DNA) and ribonucdeic acid (RNA).

Nucleic acid codes: A=adenosine; C=cytosine; G=guanosine; T=thymidine; N=equimolar A, C, G, and T; I=deoxyinosine; K=equimolar G and T; R=equimolar A and G; S=equimolar C and G; W=equimolar A and T; Y=equimolar C and T.

“Open reading frame (ORF)” refers to a region of DNA or RNA encoding a peptide, polypeptide, or protein.

“Plasmid” refers to a circular, extrachromosomal, self-replicating piece of DNA.

“Point mutation” refers to an alteration of a single nucleotide in a nucleic acid sequence.

“Polymerase chain reaction (PCR)” refers to an enzymatic technique to create multiple copies of one sequence of nucleic acid. Copies of DNA sequence are prepared by shuttling a DNA polymerase between two amplimers. The basis of this amplification method is multiple cycles of temperature changes to denature, then re-anneal amplimers, followed by extension to synthesize new DNA strands in the region located between the flanking amplimers.

“Restriction enzyme” refers to an enzyme that recognizes a specific palindromic sequence of nucleotides in double stranded DNA and cleaves both strands; also called a restriction endonuclease. Cleavage typically occurs within the restriction site.

“Vector” refers to a plasmid, cosmid, bacteriophage, or virus that carries foreign DNA into a host organism.

### DETAILED DESCRIPTION OF THE INVENTION

In a program to develop novel and improved treatment strategies for joint disease, extensive efforts were undertaken to (1) determine the ability to transduce equine synoviocytes with an adenoviral vector and quantify transgene products; (2) determine the transduction of equine synoviocytes with an adenoviral vector expressing an active equine IRAP transgene specifically as well as the ability to quantify the transgene product using commercially available methods; and (3) determine the optimal dose of equine adenoviral vector needed to produce the highest level and longest duration of transgene product with the least detriment to joint tissues.

The benefits of stable integration of the vector's genome, once advanced as a major reason to use ex vivo gene transfer, has not been realized as a clear advantage for ex vivo methods. Recent trends indicate the growing use of vectors that can be delivered in vivo as the clinical application of gene transfer approaches. In vivo administration obviates the harvesting, in vitro transduction of cells, and re-implantation to the patient that is required using ex vivo methodology. The ability to perform extensive safety testing is partially sacrificed using in vivo gene transfer, but significant safety issues have not yet been identified. Some limitations must be overcome prior to the wide spread use of gene therapy in clinical disease states. Specifically for the treatment of joint disease, the lack of long term expression and regulation of transgenes must be overcome.

The invention is directed generally towards methods to reduce the effects of joint disease in mammals. In a preferred embodiment, the method comprises selecting a mammal suspected of having a joint afflicted with a joint disease; administering one or more viral particles to the joint by arthrocentesis; and detecting a reduction in the effects of the joint disease on the treated joint; wherein the viral particles comprise a nucleic acid sequence encoding an equine interleukin-1 receptor antagonist protein. A reduction in the effects of the joint disease on the treated joint may be readily assessed by determining a lameness value between 0 and 5, according to the guidelines established by the American Association of Equine Practitioners (Anonymous Guide for veterinary service and judging of equestrian events. Lexington: AAEP, Definition and classification of lameness, 19, 1991). A physical measurement of the circumference of the joint may be used to assess a reduction in inflammation from joint disease. The mammal may generally be any mammal having joints susceptible to joint disease. More preferably, the mammal is a horse, rabbit, mouse, dog, cow, donkey, mule, or human. Most preferably, the mammal is a horse or human. A sufficient quantity of viral particles are administered to achieve a reduction in the effects of joint disease, and preferably at least about 10¹⁰ viral particles are administered to the joint. The joint disease is preferably rheumatoid or osteoarthritis. The viral particles are preferably adenoviral particles or retroviral particles, and more preferably are adenoviral particles. The nucleic acid sequence encoding an equine interleukin-1 receptor antagonist protein preferably encodes SEQ ID NO: 5, hybridizes to the reverse complement of SEQ ID NO: 4 under stringent hybridization conditions, or is at least 95% identical to SEQ ID NO: 4, and more preferably is SEQ ID NO: 4.

In an alternative embodiment, the invention is directed towards a method to produce interleukin-1 receptor antagonist protein, the method comprising: selecting a mammal comprising a joint for treatment; and administering one or more viral particles to the joint by arthrocentesis; wherein: the viral particles comprise a nucleic acid sequence encoding an equine interleukin-1 receptor antagonist protein; and the concentration of interleukin-1 receptor antagonist protein in the administered joint is higher than the concentration of interleukin-1 receptor antagonist protein an unadministered joint. The concentration of interleukin-1 receptor antagonist protein is preferably measured using a commercially available kit and manufacture's recommendation (Quantikine™ Human IL-1ra immunoassay, R & D Systems, Minneapolis, Minn.). In a non-horse mammal, the concentration of equine interleukin-1 receptor antagonist protein is directly measured; in a horse, the production of interleukin-1 receptor antagonist protein is demonstrated by an increase in concentration over the concentration in a non-treated joint. The mammal may generally be any mammal having joints susceptible to joint disease. More preferably, the mammal is a horse, rabbit, mouse, dog, cow, donkey, mule, or human. Most preferably, the mammal is a horse or human. A sufficient quantity of viral particles are administered to produce interleukin-1 receptor antagonist protein, and preferably at least about 10¹⁰ viral particles are administered to the joint. The viral particles are preferably adenoviral particles or retroviral particles, and more preferably are adenoviral particles. The nucleic acid sequence encoding an equine interleukin-1 receptor antagonist protein preferably encodes SEQ ID NO: 5, hybridizes to the reverse complement of SEQ ID NO: 4 under stringent hybridization conditions, or is at least 95% identical to SEQ ID NO: 4, and more preferably is SEQ ID NO: 4.

The invention is further useful in a preventitive or prophylactic role in delaying or preventing the onset of joint disease in a mammal. This embodiment is directed towards a method to delay or prevent the onset of joint disease in a mammal, the method comprising: selecting a mammal comprising a joint for treatment; and administering one or more viral particles to the joint by arthrocentesis; wherein the viral particles comprise a nucleic acid sequence encoding an equine interleukin-1 receptor antagonist protein. The mammal may generally be any mammal having joints susceptible to joint disease. More preferably, the mammal is a horse, rabbit, mouse, dog, cow, donkey, mule, or human. Most preferably, the mammal is a horse or human. A sufficient quantity of viral particles are administered to delay or prevent the onset of joint disease, and preferably at least about 10¹⁰ viral particles are administered to the joint. The viral particles are preferably adenoviral particles or retroviral particles, and more preferably are adenoviral particles. The nucleic acid sequence encoding an equine interleukin-1 receptor antagonist protein preferably encodes SEQ ID NO: 5, hybridizes to the reverse complement of SEQ ID NO: 4 under stringent hybridization conditions, or is at least 95% identical to SEQ ID NO: 4, and more preferably is SEQ ID NO: 4.

An additional alternative embodiment is directed towards a recombinant equine synoviocyte cell comprising a structural nucleic acid sequence encoding an equine interleukin-1 receptor antagonist protein, wherein the copy number of the structural nucleic acid sequence in the recombinant equine synoviocyte cell is higher than the copy number of the structural nucleic acid sequence in a wild type equine synoviocyte cell. Copy number of a nucleic acid sequence may be determined by Southern blotting, or by PCR. A recombinant equine synoviocyte cell comprising a structural nucleic acid sequence encoding an equine interleukin-1 receptor antagonist protein may be administered to a mammalian joint in an ex vivo treatment method. The ex vivo treatment method may be used to reduce the effects of joint disease, or may be used in a preventitive or prophylactic role. The mammal may generally be any mammal having joints susceptible to joint disease. More preferably, the mammal is a horse, rabbit, mouse, dog, cow, donkey, mule, or human. Most preferably, the mammal is a horse or human.

The Examples below use the horse and horse cells as a model system for the development and validation of this invention. The horse is an athletic species with naturally occurring joint disease, and the equine nucleic acid sequence for IRAP is known. Other mammals, including rabbit, mouse, dog, cow, donkey, mule and humans, are predicted to benefit from the IRAP gene therapy strategy due to their similarities in joint structure to the horse.

The following Examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

In Vitro Cell Culture

Synovium was aseptically harvested from grossly normal metacarpophalangeal joints of 5 horses ranging in age from 10-15 years. During processing Synovium was stored in phosphate buffered saline (PBS) at 4° C. unless otherwise noted. Synovium was further divided into 3 mm² pieces and placed in supplemented Ham's F12 media with 0.2% collagenase and cultured for 4 hours in a 37° C., 5% CO₂ environment with gentle agitation. Each liter of standard Ham's F12 was supplemented with 25 mL HEPES, 200 mL fetal calf serum, 50 mg ascorbic acid, 300 mg L-glutamine, 30 mg α-ketoglutamic acid, and 20,000 IU penicillin and streptomycin. After collagenase digestion the cell mixtures were strained using 4 ply cheesecloth and cell numbers were quantified. Cells (5×10⁵ cells/flask) were cultured in 25 cm²-culture flasks containing 4 mL of supplemented media in a 37° C. and 5% CO₂ environment. Media was changed at 7-day intervals unless otherwise noted.

### Example 2

In Vitro Transductions

All cells were grown to about 60-70% confluency before transduction experiments were started. Prior to transductions, flasks were rinsed twice with sterile phosphate buffered saline (PBS) and 2 mL of serum-free supplemented media were added to each flask. The appropriate volume of viral preparation was then added to each flask, followed by gentle rocking in a 37° C., 5% CO₂ environment for 4 hours. Cells were then rinsed twice with PBS and serum containing supplemented media added to the flasks. The cells were maintained in a 37° C., 5% CO₂ environment unless otherwise noted.

### Example 3

Synovial Fluid Analysis

Gross appearance

The appearance of the synovial fluid is noted by looking at the color and turbidity in a glass test tube. Color is rated as straw, yellow, orange, or red. Turbidity is qualitatively rated as clear, cloudy, or opaque. If iatrogenic blood contamination is observed, the sample is centrifuged for 10 minutes at 3000 rpm. This will separate the blood from the synovial fluid.

White blood cell count

Half of the synovial fluid sample is pipetted into a glass test tube. Hyaluronidase is then added to the test tube sample to decrease the viscosity. A stir stick is touched to the hyaluronidase and a small amount will remain on the stick. This amount is enough to decrease the viscosity to a “water-like” viscosity.

The hyaluronidase treated fluid is aspirated through a T660 coulter counter and the white blood cell count is reported.

Cytospin Slide Preparation

The slides are labeled with a permanent marker: horse number, joint location, cytospin, and the date. The slide, cytospin funnel and the funnel holder are put together and 2-5 drops of hyaluronidase treated fluid is added to the funnel.

The funnels are placed in the cytospin rotor and then the samples are spun for 4 minutes in the cytocentrifuge. Then slides are removed from the holders and are allowed to dry. A small circle of cells will be in the middle of the slide. When dry, the slides are stained with the dif-quick automatic stainer.

Microscopic Cytospin Slide Interpretation

A 100 cell differential of neutrophils, large mononuclear cells, lymphocytes and eosinophils is performed determining a breakdown of these four types of cells. If there are not 100 cells to count, a percentage for each type ofcell counted is calculated based on the total number of cells counted.

Total Protein

A hematocrit tube is filled with hyaluronidase treated or non-treated synovial fluid by capillary action. The fluid is then placed on the refractometer to read the specific gravity. The total protein is calculated from the specific gravity according to the following table.

TABLE 1

| Specific Gravity | Protein g/dl |
|---|---|
| 1.030 | 4.7 |
| 1.029 | 4.5 |
| 1.028 | 4.3 |
| 1.027 | 4.1 |
| 1.026 | 3.9 |
| 1.025 | 3.7 |
| 1.024 | 3.5 |
| 1.023 | 3.3 |
| 1.022 | 3.1 |
| 1.021 | 2.9 |
| 1.020 | 2.7 |
| 1.019 | 2.5 |
| 1.018 | 2.3 |
| 1.017 | 2.1 |
| 1.016 | 1.9 |
| 1.015 | 1.7 |
| 1.014 | 1.5 |
| 1.013 | 1.3 |
| 1.012 | 1.1 |
| 1.011 | 0.9 |

Mucin Clot

Several drops of synovial fluid are added to 20 ml of 5% acetic acid in a glass beaker. After 1 minute, the beaker is gently swished in a circular motion. The clump of synovial fluid can then he evaluated as good, fair or poor by dipping a wooden stir stick into the mixture and evaluating the clump by how well it holds together: Good—tight, ropy clump—holds together completely; Fair—partially breaks down at the edges; Poor—flakes and shreds—does not stay together.

### Example 4

Vectors

The adenoviral vector backbone used in this study were replication-deficient type 5 adenovirus lacking E1 and E3 loci. (Yeh, P. and Perricaudet, M., FASEB J., 11: 615-623, 1997). Construction of adenovirus vectors is well known in the art (see Hardy, S. et al., J. Virol. 71: 1842-1849, 1997). The coding region for equine IRAP (Howard, R. D., et al., Am. J. Vet. Res., 59: 712-716, 1998) was inserted in the place of the E1 region, and expression is driven by the cytomegalovirus promoter (eq-adIRAP). Previous work described the adenoviral vectors used in this study coding for the human IRAP (h-adIRAP) and β-galactosidase transgenes (adLacZ). (Nita, et al., Arthritis Rheum., 39(5): 820-828, 1996).

### Example 5

Preparation of Virus

Restriction endonuclease BamHI sites were added to the IRAP coding sequence by polymerase chain reaction (PCR) using oligonucleotide primers Horse3 (SEQ ID NO: 1) and Horse5 (SEQ ID NO: 2). The coding region of the equine RAP sequence is from nucleotides 14-548 of SEQ ID NO: 3. PCR amplification produced SEQ ID NO: 4. The amplified product was purified from a 1.5% agarose gel and ligated into the pAdlox vector (Somatix, Alameda, Calif., GenBank Accession No. U62024). The resulting plasmid was digested with restriction enzyme SfiI, and used in a co-transfection with the psi5 adenoviral backbone (Somatix Therapy Corp., Alameda, Calif.) into CRE8 cells. Plaques were isolated, expanded, and characterized for insertion of the equine IRAP cDNA and its expression. To generate stocks of virus, confluent flasks of CRE8 cells were infected with the ad.eq-IRAP virus. After detection of significant cytopathic effects, the cells were harvested, pelleted, resuspended in 5 mL of saline and stored at −80° C. To purify the virus, the cell pellet was first lysed by three rounds of freeze-thaw. The cell debris was pelleted by centrifugation, and the cleared lysate collected. Virus was banded three times over successive cesium chloride step gradients. The virus was collected after dialysis, aliquotted, and stored at −80° C.

### Example 6

Lac-Z, IRAP or PGE2 detection

Three days post adLacZ transduction cultured cells were stained with X-gal (5-bromo-4-chloroindolyl-β-D-galactose). (Nita, et al., Arthritis Rheum., 39(5): 820-828, 1996). X-gal turns a blue color when in the presence of β-galactosidase and therefore can be used as a marker of transgene production. The number of cells stained blue were counted and expressed as a percentage of the total cells counted (n=500). Concentrations of IRAP and PGE₂ were estimated from collected media stored at −80° C. Neat media aliquots were used for determination of IRAP utilizing a commercially available kit and manufacture's recommendation (Quantikine™ Human IL-1ra immunoassay, R & D Systems, Minneapolis, Minn.). Neat media aliquots were used for determination of PGE₂ utilizing a commercially available kit and manufacture's recommendation (TiterZyme® PGE₂ enzyme immunoassay kit, PerSeptive Biosystems, Inc., Framingham, Mass.).

### Example 7

In Vitro Experimental Design

Two flasks of synoviocytes were transduced with the adLacZ vector at a multiplicity of infection (MOI) of 0, 1, 10 and 100 to determine transduction frequencies. Two flasks of synoviocytes were infected with eq-as at 0, 1, 10, and 100 MOI to determine the concentration of IRAP produced. After transductions the cells were cultured for an additional 48 hours after which time the media was removed and stored (−80° C.) and the cell numbers in each flask were estimated using a hemocytometer. The concentrations of IRAP in the media samples were normalized to 1×10⁶ cells and a 48-hour culture period. To determine if equine synoviocytes transduced with the eq-adIRAP vector produced an active protein a bioassay was performed. Synoviocytes were either non-transduced or transduced (eq-adIRAP) at 10 MOI, cultured for 2 days, and then 10 ng of human recombinant Il-1α(Life Technologies, Gaithersburg, Md., USA) was added to the media of some cells followed by an additional 2 days of culture. The media was collected and stored (−80° C.) for IRAP and PGE₂ determination as well as estimations of cell numbers in each flask.

### Example 8

In Vivo Experimental Design

Six horses were used for determining concentration and duration of IRAP production following in vivo transduction of equine midcarpal and metacarpophalangeal joints at various viral doses of eq-adIRAP (ranging from 1×10⁹³-5×10¹¹ particles/joint). Each limb had one virally transduced joint while the same joint on the other limb served as the placebo treated control (receiving a similar volume of diluent). Therefore, each horse had 2 transduced joints on opposite limbs. The eq-adIRAP stock solution (1×10¹² particles/mL) was diluted in Gey's balanced salt solution for a final volume of 1000 μL. The diluent was chosen as the placebo to test the antigenicity of both the vector and proteins produced in association with its presence. Each viral dose (1×10⁹, 1×10¹⁰, 1×10¹¹, 2×10¹¹, and 5×10¹¹ particles/joint) was tested in two different horses. At the time of transduction the transduced and placebo joints were aseptically prepared and arthrocentesis performed using a 12 mL syringe and a 20 ga×1.5″ (5.08 cm) needle. Approximately 4 mL of synovial fluid was collected for routine synovial fluid analysis (total protein, WBC counts, differential cell count, color estimation, and quality of mucin clot) and IRAP quantification. The synovial fluid samples were split into tubes containing EDTA (routine synovial fluid analysis) or sodium citrate (IRAP determination). During the same procedure either the vector or placebo treatment was administered directly into the joint cavity. Arthrocenteses were repeated on days 3, 7 and at 7 day intervals thereafter until day 35 post transduction or until measured IRAP protein was similar to control levels. Previous work by others documented some difference in synovial fluid volume between the midcarpal and metacarpophalangeal joints (14.9±0.6 mL and 12.5±1.0 mL, respectively) of the-horse (Ekman, L. et al., Acta Vet. Scand. 22: 23-31, 1981). Due to this small volume difference, the synovial volumes of both joints will be considered similar for the purposes of this study.

### Example 9

In Vitro Results

Transduction efficiencies were correlated to MOI of the adLacZ vector when tested using equine synoviocytes (Table 1). Equine synoviocytes transduced with h-adIRAP demonstrated an increase in measured IRAP released into the media. Likewise, a similar increase in the concentration of IRAP measured in the media was seen after eq-adIRAP transduction of equine synoviocytes at various MOI's (FIG. 1).

Cells exposed to human IL-1α (10 ng/niL of media) for 48 hours had significantly higher PGE₂ levels in their media if they were non-transduced as compared to transduced (eq-adIRAP) cells (FIG. 2). Transduced cells exposed to IL-1α had similar PGE₂ levels to cells not exposed to IL-12α, either transduced or non-transduced transduced (FIG. 2). Similar results were observed at 100 MOI concentrations of the eq-adIRAP vector, although protection against IL-1α stimulated PGE₂ production was not seen at concentrations lower than 10 MOI.

### Example 10

In Vivo Results

When the eq-adIRAP vector was administered at a dose of 1×10⁹ particles/joint no difference in synovial fluid IRAP could be observed when placebo and transduced joints were compared. At doses of 1×10¹⁰-5×10¹¹ particles/joint, a significantly higher concentration of IRAP was detected within joints transduced with the eq-adIRAP vector compared to placebo treated joints (FIG. 3). Duration of IRAP detection within the synovial fluid of joints transduced at 2×10¹¹ particles/joint concentration was significantly longer than for joints transduced with 5×10¹¹ particles/joint (FIG. 3). An interesting finding in joints transduced at 5×10¹¹ particles/joint was a low but significant increase in measured RAP levels after day 21 post-transduction (FIG. 4). This increase was seen in both the transduced and placebo treated joints and was statistically significant at day 35 in both joints as compared to the placebo treated joints from days 0-14.

Routine clinical pathologic analysis of synovial fluids indicated that a significant decrease in the quality of mucin clot was demonstrated after repeat arthrocenteses. The mean score ± standard error on day 0 for all joints (placebo and transduced) was 1.4±0.2 compared to 2.2±0.3 on day 35. The change in mucin clot score was independent of treatment group (placebo or transduced). At the highest eq-adIRAP concentration a significantly greater influx of WBC was observed on days 3 and 7 as compared to the placebo treated joints of the same horses at a similar time period (FIG. 5). A change in the type of cell population contributing to the total WBC count was observed for different concentrations of the eq-adIRAP vector. A higher percentage of neutrophils (FIG. 6) and a lower percentage of mononuclear cells were observed in the synovial fluid of joints transduced with 0.1 and 1×10¹⁰ particles/joint. The percentages of neutrophils and mononuclear cells were independent of the sampling time period. However, on day 35 a lower percentage of the cell population was lymphocytes as compared to all other days except on day 7 where a similar trend existed, however, the statistical significance was >0.05 (FIG. 7). No differences in lymphocytes were seen with respect to concentration of the eq-adIRAP vector.

### Example 11

In Vitro Transduction Efficiency

Transduction efficiency was determined as taught by Nita, et al. (Arthritis Rheum., 39(5): 820-828, 1996). The addition of human IL-1α to the culture medium of equine synoviocytes produced a statistically significantly higher PGE₂ concentration in the media 48 hours after IL-1α administration. The transduction of synoviocytes with the eq-adIRAP vector prior to the addition of the IL-1α halted the stimulation of PGE₂ production after its addition, suggesting that the IRAP protein had significant biologic activity. Similar results were seen in a previous report using an adenoviral vector to drive the transgene expression of human IRAP in human synoviocytes and intra-articularly injected rabbits (Roessler, B. J., et al., Hum. Gene Ther., 6: 307-316, 1995) although the normalization of the data hampers further comparison. Based on the author's experience with the eq-adIRAP vector, the IRAP protein concentration in the media would have been approximately 10-20 ng/mL of media. This is only a 1-2 fold excess of IRAP compared to exogenous human IL-1α, suggesting a large excess of IRAP are not needed to combat some effects seen in response to IL-1α. The relative equal affinity of IL-1 and IRAP molecules to the IL-1 receptor has also been reported by others (Hannum, C. H. et al., Nature 343: 336-340, 1990).

TABLE 2

| MOI | Repetitions | Mean ± SEM | P-value |
|---|---|---|---|
| 0 | N = 2 | 0 ± 4.1 | N/A |
| 10 | N = 2 | 21.7 ± 4.1 | 0.0195 |
| 100 | N = 2 | 66.1 ± 4.1 | 0.0003 |
| 1,000 | N = 2 | 91.3 ± 4.1 | 0.0001 |

Table indicating the transduction efficiency (%) of the eq-adIRAP vector on equine synoviocytes. The P-value associated with the comparison of various MOI concentrations compared to the 0 MOI group is show.

### Example 12

Determination of Homologous and Degenerate Nucleic Acid Sequences

Modification and changes may be made in the sequence of the IRAP protein used in the present invention and the encoding nucleic acid sequences and still obtain a functional molecule that encodes a protein with desirable properties. The following is a discussion based upon changing the amino acid sequence of a protein to create an equivalent, or possibly an improved, second-generation molecule. The amino acid changes may be achieved by changing the codons of the nucleic acid sequence, according to the codons given in Table 3.

TABLE 3
Codon degeneracies of amino acids

| | | Three | |
|---|---|---|---|
| Amino acid | One letter | letter | Codons |
| Alanine | A | Ala | GCA GCC GCG GCT |
| Cysteine | C | Cys | TGC TGT |
| Aspartic acid | D | Asp | GAC GAT |
| Glutamic acid | E | Glu | GAA GAG |
| Phenylalanine | F | Phe | TTC TTT |
| Glycine | G | Gly | GGA GGC GGG GGT |
| Histidine | H | His | CAC CAT |
| Isoleucine | I | Ile | ATA ATC ATT |
| Lysine | K | Lys | AAA AAG |
| Leucine | L | Leu | TTA TTG CTA CTC CTG CTT |
| Methionine | M | Met | ATG |
| Asparagine | N | Asn | AAC AAT |
| Proline | P | Pro | CCA CCC CCG CCT |
| Glutamine | Q | Gln | CAA CAG |
| Arginine | R | Arg | AGA AGG CGA CGC CGG CGT |
| Serine | S | Ser | AGC AGT TCA TCC TCG TCT |
| Threonine | T | Thr | ACA ACC ACG ACT |
| Valine | V | Val | GTA GTC GTG GTT |
| Tryptophan | W | Trp | TGG |
| Tyrosine | Y | Tyr | TAC TAT |

Certain amino acids may be substituted for other amino acids in a protein sequence without appreciable loss of enzymatic activity. It is thus contemplated that various changes may be made in the peptide sequences of the disclosed protein sequences, or their corresponding nucleic acid sequences without appreciable loss of the biological activity.

In making such changes, the hydropathic index of amino acids may be considered. The importance of the hydropathic amino acid index in conferring interactive biological function on a protein is generally understood in the art (Kyte and Doolittle, J. Mol. Biol., 157: 105-132, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics. These are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate/glutamine/aspartate/asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biologically functional protein. In making such changes, the substitution of amino acids whose hydropathic indices are within ±2 is preferred, those within ±1 are more preferred, and those within ±0.5 are most preferred.

It is also understood in the art that the substitution of like amino acids may be made effectively on the basis of hydrophilicity. U.S. Pat. No. 4,554,101 (Hopp, T. P., issued Nov. 19, 1985) states that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0 ±1); serine (+0.3); asparagine/glutamine (+0.2); glycine (0); threonine (−0.4); proline (−0.5 ±1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4).

It is understood that an amino acid may be substituted by another amino acid having a similar hydrophilicity score and still result in a protein with similar biological activity, i.e., still obtain a biologically functional protein. In making such changes, the substitution of amino acids whose hydropathic indices are within ±2 is preferred, those within ±1 are more preferred, and those within ±0.5 are most preferred.

As outlined above, amino acid substitutions are therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine, and isoleucine. Changes which are not expected to be advantageous may also be used if these resulted in functional proteins.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention.

1. A method to reduce the effects of joint disease in a mammal, the method comprising:
selecting a mammal suspected of having a joint afflicted with a joint disease;
administering one or more viral particles to the joint by arthrocentesis; and
detecting a reduction in the effects of the joint disease on the treated joint;
wherein the viral particles comprise a nucleic acid sequence encoding an equine interleukin-1 receptor antagonist protein.

2. The method of claim 1, wherein the mammal is a horse.

3. The method of claim 1, wherein the mammal is a rabbit, mouse, dog, cow, donkey or mule.

4. The method of claim 1, wherein the mammal is a human.

5. The method of claim 1, wherein at least about 10⁹ viral particles are administered to the joint.

6. The method of claim 1,wherein the viral particles are adenoviral particles.

7. A method to increase the influx of white blood cells into the joint of a mammal, the method comprising:
selecting a mammal comprising a joint for treatment; and
administering one or more viral particles to the joint by arthrocentesis;
wherein:
the viral particles comprise a nucleic acid sequence encoding an equine interleukin-1 receptor antagonist protein; and
the rate of influx of white blood cells into the administered joint is higher than the influx of white blood cells in an untreated joint.

8. The method of claim 7, wherein the mammal is a horse.

9. The method of claim 7, wherein the mammal is a rabbit, mouse, dog, cow, donkey, or mule.

10. The method of claim 7, wherein the mammal is a human.

11. The method of claim 7, wherein at least about 10⁹ viral particles are administered to the joint.

12. The method of claim 7, wherein the viral particles are adenoviral particles.

13. A method to increase the percentage of neutrophils in the joint of a mammal, the method comprising:
selecting a mammal comprising a joint for treatment; and
administering one or more viral particles to the joint by arthrocentesis;
wherein:
the viral particles comprise a nucleic acid sequence encoding an equine interleukin-1 receptor antagonist protein; and
the percentage of neutrophils in the administered joint is higher than the percentage of neutrophils in an untreated joint.

14. The method of claim 7, wherein the mammal is a horse.

15. The method of claim 7, wherein the mammal is a rabbit, mouse, dog, cow, donkey, or mule.

16. The method of claim 7, wherein the mammal is a human.

17. The method of claim 7, wherein at least about 10⁹ viral particles are administered to the joint.

18. The method of claim 7, wherein the viral particles are adenoviral particles.

19. A method to decrease the percentage of mononuclear cells in the joint of a mammal, the method comprising:
selecting a mammal comprising a joint for treatment; and
administering one or more viral particles to the joint by artirocentesis;
wherein:
the viral particles comprise a nucleic acid sequence encoding an equine interleukin-1 receptor antagonist protein; and
the percentage of mononuclear cells in the administered joint is lower than the percentage of mononuclear cells in an untreated joint.

20. The method of claim 19, wherein the mammal is a horse.

21. The method of claim 19, wherein the mammal is a rabbit, mouse, dog, cow, donkey, or mule.

22. The method of claim 19, wherein the mammal is a human.

23. The method of claim 19, wherein at least about 10⁹ viral particles are administered to the joint.

24. The method of claim 19, wherein the viral particles are adenoviral particles.

25. A method to produce interleukin-1 receptor antagonist protein in vivo, the method comprising:
selecting a mammal comprising a joint for treatment; and
administering one or more viral particles to the joint by arthrocentesis;
wherein:
the viral particles comprise a nucleic acid sequence encoding an equine interleukin-1 receptor antagonist protein; and
the concentration of interleukin-1 receptor antagonist protein in the administered joint is higher than the concentration of interleukin-1 receptor antagonist protein in an unadministered joint.

26. The method of claim 25, wherein the mammal is a horse.

27. The method of claim 25, wherein the mammal is a rabbit, mouse, dog, cow, donkey, or mule.

28. The method of claim 25, wherein the mammal is a human.

29. The method of claim 25, wherein at least about 10⁹ viral particles are administered to the joint.

30. The method of claim 25, wherein the viral particles are adenoviral particles.

31. A method to delay the onset of joint disease in a mammal, the method comprising:
selecting a mammal comprising a joint for treatment; and
administering one or more viral particles to the joint by arthrocentesis;
wherein the viral particles comprise a nucleic acid sequence encoding an equine interleukin-1 receptor antagonist protein.

32. The method of claim 31, wherein the mammal is a horse.

33. The method of claim 31, wherein the mammal is a rabbit, mouse, dog, cow, donkey, or mule.

34. The method of claim 31, wherein the mammal is a human.

35. The method of claim 31, wherein at least about 10⁹ viral particles are administered to the joint.

36. The method of claim 31, wherein the viral particles are adenoviral particles.
